(19) United States
(12) Patent Application Publication
(10) Pub. No.: US 2025/0255555 A1
(43) Pub. Date: Aug. 14, 2025

(54) SELF-POWERED BIOELECTRONIC STENT SENSOR SYSTEM, DEVICE AND METHOD

(71) Applicant: THE REGENTS OF THE UNIVERSITY OF CALIFORNIA, Oakland, CA (US)

(72) Inventors: Jun Chen, Los Angeles, CA (US); Geoffrey P. Colby, Santa Cruz, CA (US); Guorui Chen, Los Angeles, CA (US); Anthony C. Wang, Los Angeles, CA (US); Paul S. Weiss, Los Angeles, CA (US)

(21) Appl. No.: 18/881,905

(22) PCT Filed: Jul. 28, 2023
(86) PCT No.: PCT/US23/71193

§ 371 Date: Jan. 7, 2025

Related U.S. Application Data
(60) Provisional application No. 63/369,986, filed on Aug. 1, 2022.

(51) Int. Cl. A61B 5/00; A61B 5/02

(52) U.S. Cl. CPC A61B 5/6862; A61B 5/0022; A61B 5/02; A61B 2562/028

(57) ABSTRACT

A bioelectronic stent sensor system comprises a bioelectronic stent sensor device comprising a first hollow cylindrical lattice, and a second hollow cylindrical lattice attached to a first surface of the first lattice, comprising a biocompatible magnetoelastic micromesh (BMM), and a computing system communicatively connected to the bioelectronic stent sensor device, comprising a processor and a non-transitory computer-readable medium with instructions stored thereon, which when executed by a processor, perform steps comprising receiving readout current signals from the bioelectronic stent sensor device, and calculating a blood flow rate based on the readout current signals by establishing an empirical relationship between the readout current signals and a flow rate value. Related devices and methods are also disclosed.

### CROSS-REFERENCE TO RELATED APPLICATIONS

This application claims priority to U.S. provisional application No. 63/369,986 filed on Aug. 1, 2022, incorporated herein by reference in its entirety.

### BACKGROUND OF THE INVENTION

Diseases caused by atherosclerosis, such as myocardial infarction, stroke, and peripheral vascular disease, are the leading cause of death in the United States, affecting over 4.6 million people.¹ Angioplasty and stent placement,² the procedure of implanting an expandable metal stent to open stenosed, or partially blocked arteries (FIG. 1A), is routinely used to treat atherosclerosis in various locations (e.g., carotid arteries and coronary arteries) and where the stent can be implanted via balloon catheter or as a self-expanding stent.³ After each of these operations, ensuring blood flow through the newly expanded artery is critical.⁴ However, in the ensuing months after stenting, up to 41% of cases witness in-stent re-stenosis (ISR), due to excess growth of endothelial tissue, inflammatory scarring, and/or new plaque.⁵ This could result in life-threatening heart problems such as unstable angina, acute coronary syndrome, and heart attacks. Current diagnostic modalities for in-stent restenosis are angiography⁶ and duplex ultrasound,⁷ which are intermittent, highly operator-dependent, labor-intensive, time-consuming, and/or invasive. In some cases, in-stent restenosis is not noted until the opportunity to save the angioplasty has passed,⁸ making repeated intervention a requirement and more difficult. Stent construction supplemented with embedded blood flow sensing capability would enable continuous real-time detection of in-stent re-stenosis without the need for additional diagnostic procedures.⁹ To date, flow sensor development has been limited by bulky structures due to the necessary encapsulation layer and power source, which hinder application in small blood vessels.¹⁰ Meanwhile, many wireless flow sensors rely on radio-frequency inductive coupling to enable interrogation, and readouts can be inaccurate and influenced by positional variations between the devices and the external radio frequency excitation source.¹¹ They need an external antenna as a “power source” to interact with the sensor to induce a resonance that is responsive to the hemodynamics, which are complex, inaccurate, and susceptible to positional variations.¹²⁻¹⁴ Thus, there is a major unmet need to monitor the hemodynamic variations after angioplasty and to diagnose in-stent restenosis in an accurate, reliable, continuous, noninvasive, timely, and easily accessible manner.

### SUMMARY OF THE INVENTION

Some embodiments of the invention disclosed herein are set forth below, and any combination of these embodiments (or portions thereof) may be made to define another embodiment.

In one aspect, a bioelectronic stent sensor device comprises a first hollow cylindrical lattice, and a second hollow cylindrical lattice attached to a first surface of the first lattice, comprising a biocompatible magnetoelastic micromesh (BMM).

In one embodiment, the first surface of the first lattice comprises an inner surface. In one embodiment, the first lattice comprises a metal lattice. In one embodiment, the metal lattice includes a layer of nanoparticles, metal nanoparticles, noble metal nanoparticles, or gold nanoparticles. In one embodiment, the BMM comprises a plurality of nanomagnets embedded in a polymer matrix. In one embodiment, the second lattice includes a plurality of microstructures in an array. In one embodiment, the microstructures comprise pyramids, cylinders, or hemispheres. In one embodiment, the microstructures have a lateral length of 100 nm to 5000 nm, and a pitch of 1 μm to 1000 μm. In one embodiment, the array is lithographically patterned. In one embodiment, the device further comprises an antenna attached to the first lattice. In one embodiment, the antenna is attached via laser microwelding. In one embodiment, the second lattice is attached to the first lattice via a cyanoacrylate instant adhesive or a catechol-based adhesive. In one embodiment, the first lattice comprises a double layer lattice, and wherein the second lattice is anchored between the layers of the first lattice. In one embodiment, the BMM is configured to deform and to shift its magnetic flux to induce a current in the first lattice. In one embodiment, the BMM comprises a polymer and magnetic nanoparticle composite. In one embodiment, the polymer comprises an Ecoflex rubber and the magnetic nanoparticle composite comprises NdFeB microparticles and/or nanoparticles. In one embodiment, a nanoparticle layer surrounds the magnetic nanoparticle composite. In one embodiment, the surrounding nanoparticle layer comprises SiO₂ nanoparticles (see Zhao X, Chen G, Zhou Y, Nashalian A, Xu J, Tat T, Song Y, Libanori A, Xu S, Li S, Chen J. Giant Magnetoelastic Effect Enabled Stretchable Sensor For Self-Powered Biomonitoring. ACS Nano. 2022; 16 (4): 6013-6022). In one embodiment, the device has at least one of: a sensitivity detection limit of less than 0.5 cm/second, a low flow rate detection limit of less than 0.5 cm/second, a short response time of less than 15 ms, a high signal-to-noise ratio (SNR) of greater than 50 dB, and/or long-term stability.

In another aspect, a bioelectronic stent sensor system comprises a bioelectronic stent sensor device comprising a first hollow cylindrical lattice, and a second hollow cylindrical lattice attached to a first surface of the first lattice, comprising a biocompatible magnetoelastic micromesh (BMM), and a computing system communicatively connected to the bioelectronic stent sensor device, comprising a processor and a non-transitory computer-readable medium with instructions stored thereon, which when executed by a processor, perform steps comprising receiving readout current signals from the bioelectronic stent sensor device, and calculating a blood flow rate, a pressure, a pulse rate, an embolic event, a vessel stiffness or change thereof, an oxygenation, and/or a particle in the flow based on the readout current signals via an established empirical relationship between the readout current signals and a flow rate value.

In one embodiment, the bioelectronic stent sensor device is wirelessly communicatively connected to the computing system via a wireless communication protocol comprising 3G, 4G/LTE, 5G, 6G, Bluetooth, Bluetooth Low Energy (BLE), Zigbee, or near-field communication (NFC).

In another aspect, a blood flow rate monitoring method comprises providing the bioelectronic stent sensor system as described above, implanting the bioelectronic stent sensor device via a standard stent placement procedure, receiving readout current signals from the bioelectronic stent sensor device on the computing system, and calculating a blood flow rate, a pressure, a pulse rate, an embolic event, a vessel stiffness or change thereof, an oxygenation, or a particle in the flow based on the readout current signals via an established empirical relationship between the readout current signals and a flow rate value.

In one embodiment, finite element analysis (FEA) is used to establish the empirical relationship between the readout current signals and the flow rate value.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A depicts an exemplary procedure of implanting a traditional metal stent to open stenosed or partially blocked arteries in accordance with some embodiments.

FIG. 1B depicts an exemplary bioelectronic stent sensor based on the giant magnetoelastic effect configured to enable postoperative blood flow rate monitoring in accordance with some embodiments.

FIG. 2A depicts a first view of an exemplary bioelectronic stent sensor including a biocompatible magnetoelastic micromesh (BMM) lattice attached to the inner wall of a metal lattice in accordance with some embodiments.

FIG. 2B depicts additional views of an exemplary bioelectronic stent sensor including a biocompatible magnetoelastic micromesh (BMM) pressure sensitive lattice attached to the inner wall of a metal lattice in accordance with some embodiments.

FIG. 2C depicts an exemplary Ecoflex rubber/NdFeB nanoparticle composite in accordance with some embodiments.

FIG. 2D depicts how pulsatile blood flow deforms the patterned BMM and shifts its magnetic flux in accordance with some embodiments.

FIG. 2E depicts how ISR leads to a perturbation in blood flow which can be detected by the smart stent in accordance with some embodiments.

FIG. 3A depicts an exemplary experimental device where a soft MEG was wrapped around the carotid artery to detect the artificial blood flow rate in accordance with some embodiments. (Scale bar is 3 mm)

FIG. 3B depicts an exemplary experimental Masson's Trichrome staining of histological sections of the control sample after 14 days in accordance with some embodiments. (Scale bar is 100 μm)

FIG. 3C depicts an exemplary experimental Masson's Trichrome staining of histological sections of the soft MEG sample after 14 days in accordance with some embodiments. (Scale bar is 100 μm)

FIG. 3D is a plot showing exemplary experimental current signals induced by different flow rates in accordance with some embodiments.

FIG. 3E is a plot showing exemplary experimental current signals which reflect the experimentally induced vessel occlusion in accordance with some embodiments.

FIG. 4A depicts an exemplary experimental biocompatible magnetoelastic micromesh (BMM) with dimensions of 15 mm by 15 mm by 0.1 mm, comprised of Ecoflex rubber/NdFeB nanoparticle composite in accordance with some embodiments. (Scale bar is 5 mm).

FIG. 4B depicts an exemplary experimental bioelectronic stent sensor (25 mm length, 5.6 mm diameter, and 0.3 mm thickness) including a biocompatible magnetoelastic micromesh (BMM) lattice attached to the inner wall of a metal lattice in accordance with some embodiments. (Scale bar is 4 mm).

FIG. 4C depicts an exemplary experimental bioelectronic stent sensor (25 mm length, 5.6 mm diameter, and 0.3 mm thickness) including a biocompatible magnetoelastic micromesh (BMM) lattice attached to the inner wall of a metal lattice in accordance with some embodiments. (Scale bar is 2 mm).

FIG. 4D is a plot showing exemplary experimental current signals generated by the bioelectronic stent sensor which reflect the experimentally induced vessel occlusion in accordance with some embodiments.

FIGS. 5A-5C depicts an exemplary smart stent fabrication procedure in accordance with some embodiments.

FIG. 5D depicts an exemplary surface magnetic flux density mapping in accordance with some embodiments.

FIG. 5E depicts an exemplary strain stress curve of pressure sensitive micromesh (BMM) in accordance with some embodiments.

FIG. 5F depicts an exemplary device that has been micro-welded to tightly bind the pressure sensitive micromesh and metal stent in accordance with some embodiments. Scale bar is 0.8 mm.

FIGS. 5G-5H depict bending and twisting of the device of FIG. 5F without causing any divorces in accordance with some embodiments. Scale bars are 3 mm.

FIG. 5I depicts various smart stent devices configured to function on different blood vessels. Scale bar is 3 mm.

FIG. 6A depicts an experimental setup for testing the flow sensing ability of a smart stent in accordance with some embodiments.

FIG. 6B depicts example current waveforms induced by different pulse waves. I₁ and I₂ are current features and t₁ and t₂ are time features acquired from the waveforms in accordance with some embodiments.

FIG. 6C depicts dependence of the electrical output and pulse to pulse time of the smart stent on different heart rates in accordance with some embodiments.

FIG. 6D depicts current outputs induced by different flow rates in accordance with some embodiments.

FIG. 6E depicts a linear relationship between the induced current signals and different flow rates in accordance with some embodiments.

FIG. 6F depicts how magnetic fields are transparent to the tissue and can effectively reach the receiver in accordance with some embodiments.

FIG. 6G depicts current signals reflecting the experimentally induced vessel occlusion in accordance with some embodiments.

FIG. 6H depicts dependence of the current outputs on different blocking branches in accordance with some embodiments.

FIG. 6I depicts a cyclic test of the smart stents for more than 2.7×10⁶ cycles.

FIG. 7 depicts an exemplary computing environment in which aspects of the systems, devices and methods of self-powered bioelectronic stent sensors may be practiced in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems, devices and methods of self-powered bioelectronic stent sensors. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are self-powered bioelectronic stent sensor systems, devices, and methods.

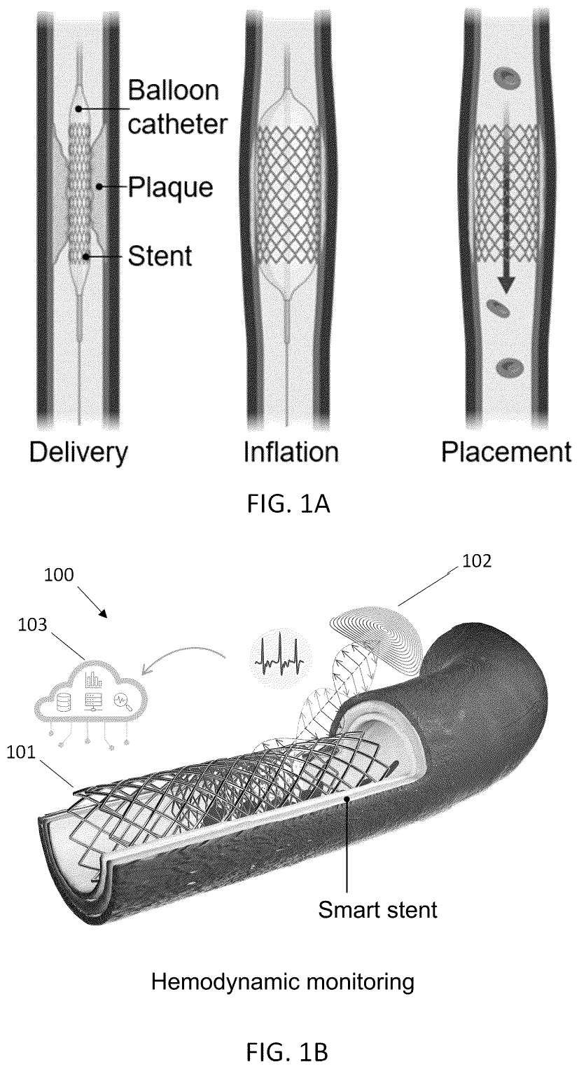

To address the abovementioned challenges, disclosed herein is a smart bioelectronic stent sensor device 101 and system 100 based on the giant magnetoelastic effect that enables postoperative blood flow rate monitoring and ISR diagnosis with self-powered, accurate, real-time, miniaturized, and biocompatible properties (FIG. 1B). The giant magnetoelastic effect in soft matter has been shown in use in a soft magnetoelastic generator (MEG) as a novel type of self-powered pressure sensor for cardiovascular monitoring.¹⁶⁻¹⁸ Since magnetic fields can pass through water with negligible intensity loss, the giant magnetoelastic effect-based bioelectronics is intrinsically waterproof. The Live/Dead assay of human fibroblasts being cultured on the soft MEG confirmed its high biocompatibility. MEG-based pressure sensors have been developed to convert diverse types of biomechanical motion, including pulse wave,¹⁶ respiration, ¹⁷ and heart beat¹⁸ into real-time electrical signals featuring intrinsically waterproof, self-powered, and biocompatible properties. These superiorities make the soft MEG a useful technology to develop the bioelectronic stent sensor 101 for postoperative blood flow rate monitoring.

FIG. 1B depicts an exemplary bioelectronic stent sensor system 100 including a bioelectronic stent sensor device 101 comprising a first hollow cylindrical lattice 104, and a second hollow cylindrical lattice 105 attached to a first surface of the first lattice 104. In some embodiments, the system 100 comprises a core-shell configuration. In some embodiments, the first lattice 104 and/or the second lattice 105 are stents. In some embodiments, the second lattice 105 comprises a biocompatible magnetoelastic micromesh (BMM) which can be used for example, for hemodynamic sensing and timely diagnosis of re-stenosis. The system 100 further includes a computing system 102 communicatively connected to the bioelectronic stent sensor device, comprising a processor and a non-transitory computer-readable medium with instructions stored thereon, which when executed by a processor, perform steps comprising receiving readout current signals from the bioelectronic stent sensor device 101, and calculating a blood flow rate based on the readout current signals via an established empirical relationship between the readout current signals and a flow rate value.

In some embodiments, the bioelectronic stent sensor device 101 is magnetically responsive. In some embodiments, the bioelectronic stent sensor device 101 is compatible with standard microcatheters. In some embodiments, external magnetic manipulation can be used to navigate the smart stent through the complex vasculature with ease during deployment.

In some embodiments the bioelectronic stent sensor device 101 is wirelessly communicatively connected to the computing system 102 via a wireless communication protocol such as 3G, 4G/LTE, 5G, Bluetooth, Bluetooth Low Energy (BLE), Zigbee, near-field communication (NFC), or any other suitable wireless connection protocol. In some embodiments, the bioelectronic stent sensor device 101 is communicatively connected to the computing system 102 via a wired communication protocol such as ethernet, USB, fiberoptic, or any other suitable wired connection protocol. In some embodiments, the computing system 102 is communicatively connected via wireless and/or wired connection protocols to a network 103. In some embodiments, the network 103 can be configured as a clinical data cloud.

In some embodiments, the first surface of the first lattice 104 comprises an inner surface or inner wall. In some embodiments, the first lattice 104 and/or the second lattice 105 are substantially cylindrical. In some embodiments, the first lattice 104 is a metal lattice or a non-metallic polymer lattice, and the second lattice 105 is a BMM lattice. In some embodiments, the first lattice 104 comprises a metal lattice which can include a layer of nanoparticles configured to increase the conductance of the lattice. In some embodiments, the nanoparticles comprise metal nanoparticles, noble metal nanoparticles, and/or gold nanoparticles.

In some embodiments, the length of the device 101, first lattice 104, and/or second lattice 105 is 0.1 mm to 10 cm or 2 mm to 5 cm, the expanded diameter is 0.1 mm to 10 cm or 2 mm to 1.5 cm, the compressed diameter is 0.1 mm to 10 cm or 0.1 mm to 5 mm, the wall thickness is 0.0001 mm to 10 mm or 0.05 mm to 3 mm, and the crowns are 0.001 mm to 10 mm or 0.001 mm to 0.2 mm wide and/or thick, or any other suitable dimensions.

In some embodiments, such as intracranial metal lattices, the length of the metal lattice 104 is 7 mm to 48 mm, the expanded diameter is 2 mm 8 mm, the compressed diameter is 0.5 mm to 0.9 mm, and the crowns are 0.05 mm to 0.095 mm wide or thick. In some embodiments, for example stents used for larger arteries like the aorta and veins like the vena cava, the dimensions of the metal lattice 104 are about 3 cm or about 5 cm. Example material options for the metal lattice include Fe, Fe-35Mn alloy, Fe-10Mn-1Pd alloy, Fe alloyed by different elements (Mn, Co, Al, W, Sn, B, C, and S) as cast, Fe-30Mn-6Si alloy, or other suitable materials.

In some embodiments, the BMM 105 has a length of 1 mm to 48 mm, an expanded diameter of 1.0 mm to 4.5 mm, a compressed diameter of 0.1 mm to 0.9 mm, and a wall thickness of 0.001 mm to 0.4 mm, or any other suitable dimensions which can be adapted based on the size of the metal stent used.

The voltage and current generated by the device 101 is based on the device dimensions and materials used, and in some examples the voltages ranges from 1 pV to 1 V and the current ranges from 1 pA to 1 A.

In some embodiments, the BMM second lattice 105 comprises a plurality of nanomagnets 107 embedded in a polymer matrix 106. In some embodiments, the second lattice 105 includes a plurality of pyramid structures in an array. In some embodiments, the pyramid structures have a lateral length of 100 nm to 5000 nm or 500 nm to 5000 nm, and a pitch of 1 μm to 1000 μm, and can be lithographically patterned. In some embodiments, the BMM is configured to deform and shift its magnetic flux to induce a current in the first lattice 104. In some embodiments, the BMM comprises a polymer and magnetic nanoparticle composite. In some embodiments, the BMM comprises an Ecoflex rubber and NdFeB nanoparticles composite. In some embodiments, a nanoparticle layer surrounds the magnetic nanoparticle composite. In some embodiments, the surrounding nanoparticle layer comprises SiO₂ nanoparticles. In some embodiments, a coating of a SiO₂ nanoparticles layer surrounds the NdFeB nanoparticles.

In some embodiments, an antenna attached to the first lattice 104 via a laser microwelding or other suitable process. In some embodiments, the second lattice 105 is attached to the first lattice 104 via a cyanoacrylate instant adhesive, a catechol-based adhesive such as one based on polydopamine, or other suitable mechanism.

In some embodiments, the first lattice 104 comprises a double layer lattice. In some embodiments, the second lattice 105 is anchored between the layers of the first lattice 104.

In some embodiments, the device 101 has a sensitivity detection limit of less than 0.5 cm/second, a low flow rate detection limit of less than 0.5 cm/second, a short response time of less than 15 ms, a high signal-to-noise ratio (SNR) of greater than 50 dB, and long-term stability.

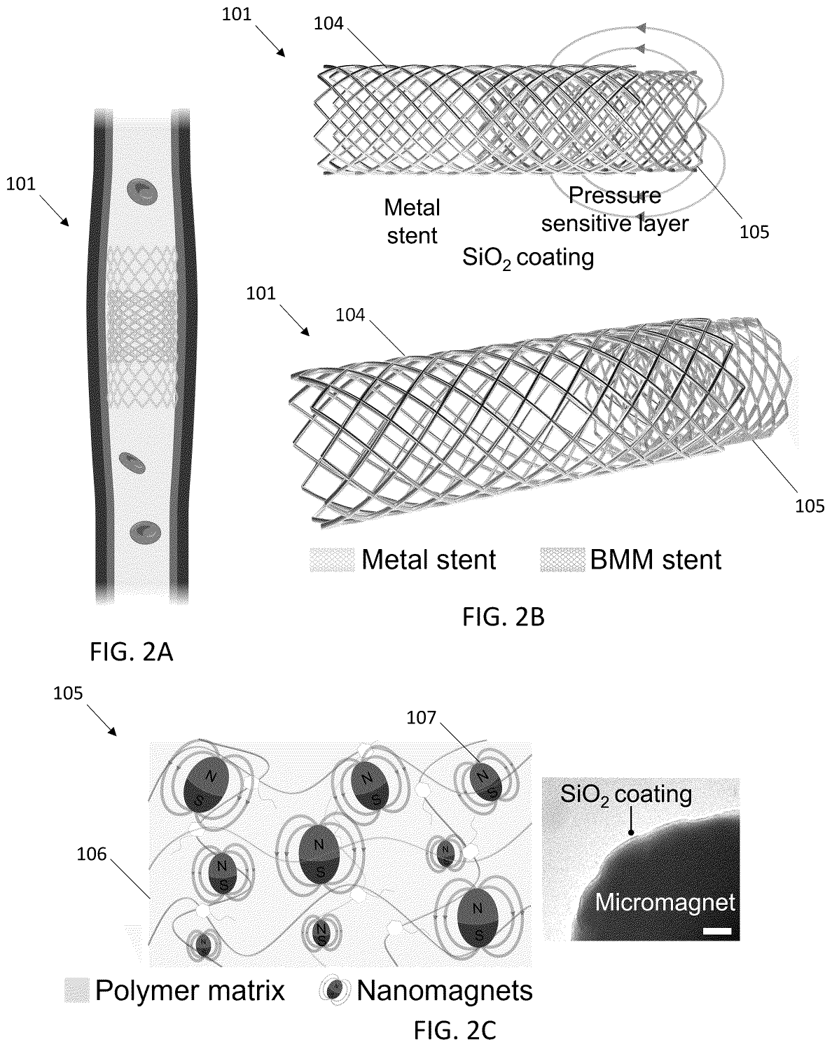

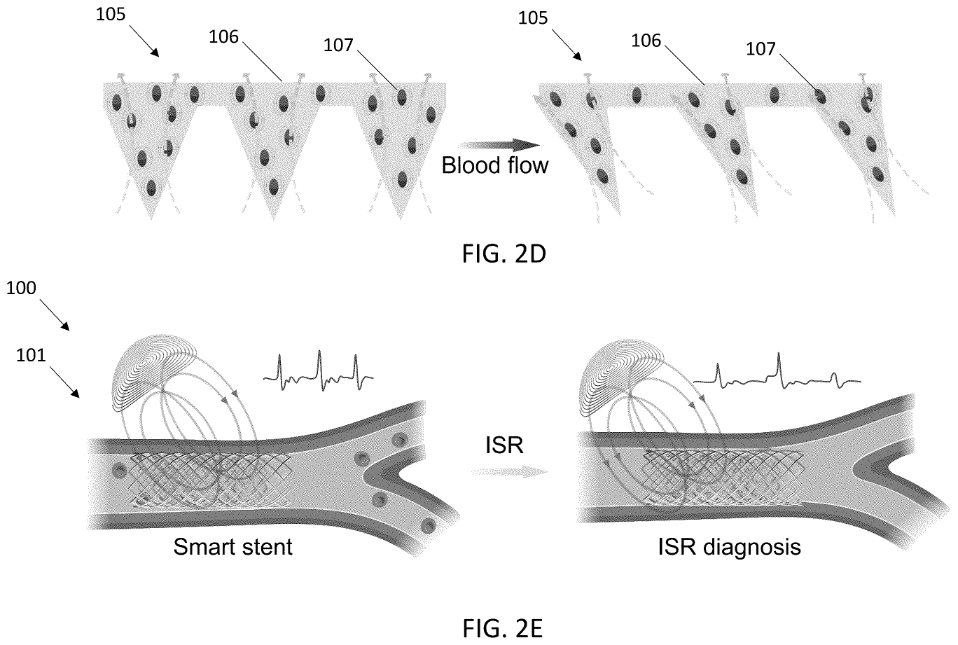

As shown in FIG. 2A, in some embodiments, the exemplary bioelectronic stent sensor 101 includes a biocompatible magnetoelastic micromesh (BMM) (FIGS. 2B and 2C) lattice 105 attached to the inner wall of a highly conductive metal lattice 104. In some embodiments, the BMM includes a lithographically patterned surface array. Further details on the BMM material are described in Y. Zhou et al. (Giant Magnetoelastic Effect in Soft Systems for Bioelectronics. Nat. Mater. 2021, 20, 1670-1676), incorporated herein by reference in its entirety.

Pulsatile blood flow can deform the BMM lattice 105 and shift its magnetic flux (FIG. 2D), thus inducing a current in the metal lattice 104. Assisted by computational modeling techniques, blood flow rates, pressure, pulse rate, embolic events, vessel stiffness or change thereof, oxygenation, and/or a particle in the flow can be calculated from these recorded current signals, which can serve as a biomarker for timely in-stent restenosis diagnosis and management. In some embodiments, a finite element analysis can be used to establish an empirical relationship between the readout current signals (nA) and the real value of flow rate (cm/second). In some embodiments (FIG. 2E), since ISR leads to a perturbation in blood flow, which can then be detected and/or diagnosed by the system 100.

In some embodiments, for improved sensitivity and response time of the bioelectronic stent sensor device 101, the BMM lattice 105 includes a microstructured structure array on its surface. This microstructure allows the BMM lattice 105 to deform elastically when systolic/diastolic blood pressure is applied, shifting the magnetic field reversibly, thus maximizing the generated signals. Two parameters of the structures are closely tied to BMM performance, the lateral length, and the pitch.

In some embodiments, large-area periodic inverted pyramid templating structures are utilized in creation of the pyramid structures. In some embodiments, the dimensions of the pyramid structures are based on COMSOL simulation results. In some embodiments, the pyramid structures can have a lateral length of 100 nm to 5000 nm, 200 nm to 1500 nm, 250 nm to 1000 nm, about 1000 nm, about 500 nm, about 250 nm, or any other suitable length. In some embodiments, the pyramid structures can have a pitch of 1 μm to 1000 μm, 1 μm to 10 μm 5 μm to 10 μm, about 5 μm, about 10 μm, or any other suitable pitch. In some embodiments, the pyramid structures are manufactured on the PDMS thin film as the templating substrates via nanosphere lithography.²⁰⁻²² In some embodiments, spin-casting an uncured polymer/magnetic nanoparticle composite solution through an inverted microstructure templating substrate is used to fabricate magnetoelastic thin films (200 μm thick) with different microstructured surfaces. In some embodiments, spin-casting uncured Ecoflex rubber/NdFeB nanoparticle (5 nm) composite solution through the inverted-pyramid templating substrates is used to fabricate magnetoelastic thin films (200 μm thick) with different microstructured surfaces. Other microstructures that can be utilized include cylinders and hemispheres with a lateral length of 100 nm to 5000 nm and a pitch of 1 μm to 10 μm.

In some embodiments, the bioelectronic stent sensor device 101 offers attractive features such as the elimination of the requirement of external radiofrequency excitation and realize accurate, reliable, and continuous monitoring of blood flow rate. Furthermore, in some embodiments, since magnetic fields can pass through water with negligible intensity loss, the giant magnetoelastic effect-based bioelectronic stent sensor is intrinsically waterproof, which avoids the pitfalls of bulky and rigid encapsulation layers and miniaturizes the total system. In some embodiments, the innovative dual-layer construction adds blood flow sensing capabilities without compromising the original expanding functionality of the stent, and the micromesh structure avoids occlusion of covered side branches, enabling stent implantation in widespread anatomy/organs including the brain, coronary, carotid, and peripheral vasculature. In some embodiments, the device 101 can be configured to be positioned in or around a vessel (e.g, artery or vein) or configured as a non-stent sensor device configured to be positioned in or around a mobile organ (e.g., heart).

In some embodiments, gold nanoparticles are sputtered onto the metal lattice 104 to achieve high conductivity for generating current signals induced by the BMM lattice 105. In some embodiments, laser microwelding is used to integrate an ultrathin antenna chip (for example 3×6 mm², 200 μm thick) with the metal lattice 104 robustly to transmit the generated signals. In some embodiments, to construct the bioelectronic stent sensor device 101, the optimal BMM is bent to mimic the stent strut design and inserted into the metal lattice 105.

In some embodiments, medical-grade cyanoacrylate instant adhesive (for example Adhesive Systems, MG30) is used to adhere the BMM lattice 105 securely to the inner wall of the metal lattice 104. This mechanically robust bonding is able to withstand deformation without dislodging the BMM lattice 105 during device delivery. In some embodiments, to validate the bonding a calibration electrodynamic transducer (for example Labworks, ET-126HF) is used to compress the bioelectronic stent sensors 100,000 times. With a simple reconfiguration, e.g., changing the diameter of the metal lattice 104, the devices 101 can be adapted to function on different blood vessels. In some embodiments, a double-layer metal lattice can be used as the substrate to anchor the BMM/chip mechanically between the two layers to prevent dislodging of the BMM/chip by the catheter.²⁷

In some embodiments, the bioelectronic stent sensor device 101 is configured to have sensitivity and low flow rate detection limits of less than 0.5 cm/second, short response times of less than 15 ms, high signal-to-noise ratio (SNR) of greater than 50 dB, and/or long-term stability (i.e. no obvious attenuation). In some embodiments, a nanoparticle coating layer, such as SiO₂ for example, is coated around the magnetic nanoparticles, such as NdFeB for example, to ensure even distribution inside the polymer matrix, especially in the surface microstructures.¹⁸|

In some embodiments, the bioelectronic stent sensor devices 101 are compatible with the standard displacement/implantation procedure during angioplasty, which eliminates the need for another surgery for deployment/removal. In some embodiments, the device 101 is delivered and deployed in an anatomical lumen via a delivery catheter.

In some embodiments, a blood flow rate monitoring method begins with providing the bioelectronic stent sensor system 100 including the bioelectronic stent sensor device 101. The bioelectronic stent sensor device is then implanted via a standard stent placement procedure. Readout current signals from the bioelectronic stent sensor device are then received on the computing system. The readout current signals are created by pulsatile blood flow deforming the BMM lattice 105 changing the magnetic field, which then induces a current in the metal lattice 104. An antenna attached to the metal lattice 104 is configured to transmit the readout current signals. A blood flow rate is then calculated and displayed based on the readout current signals via an established empirical relationship between the readout current signals and a flow rate value. In some embodiments, finite element analysis (FEA) is used to establish the empirical relationship between the readout current signals and the flow rate value.

In some embodiments, the BMM 105 is positioned on an inner endoluminal surface proximate to the bloodstream. In some embodiments, the BMM 105 is positioned on an outer surface proximate to the inner vessel wall. In some embodiments, the BMM 105 is positioned between two or more stent layers. In some embodiments, the BMM 105 is woven within the metal lattice stent 104. In some embodiments, the device 100 comprises only the BMM 105. In some embodiments, the BMM 105 is positioned along the full length, in a proximal portion only, in a distal portion only, and/or in a central portion only with respect to the metal lattice stent 104.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention, and are not to be construed as limiting in any way the remainder of the disclosure.

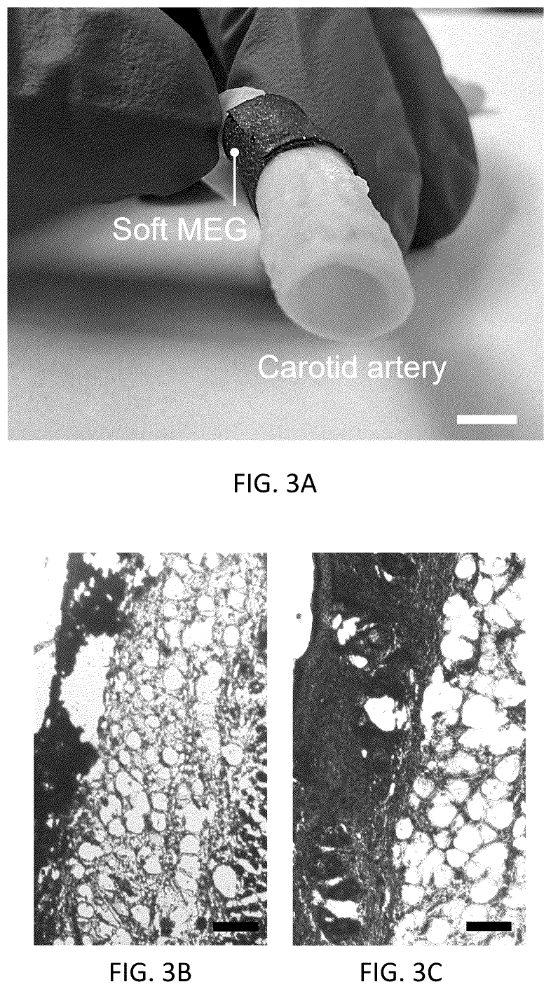

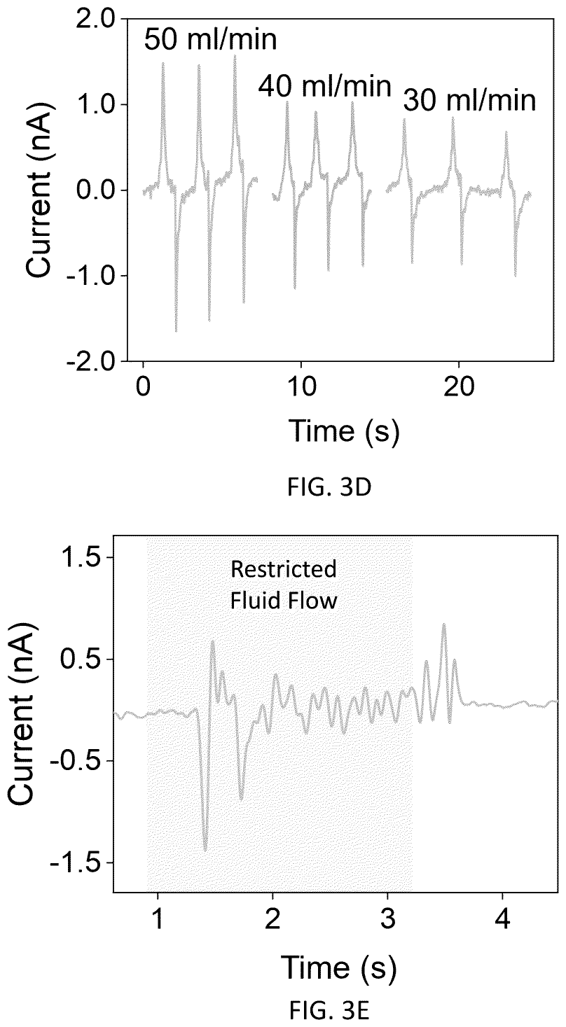

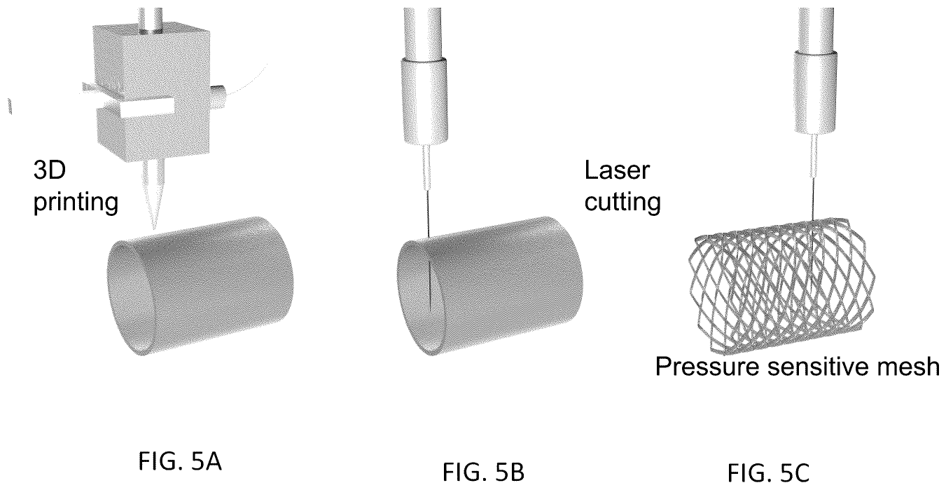

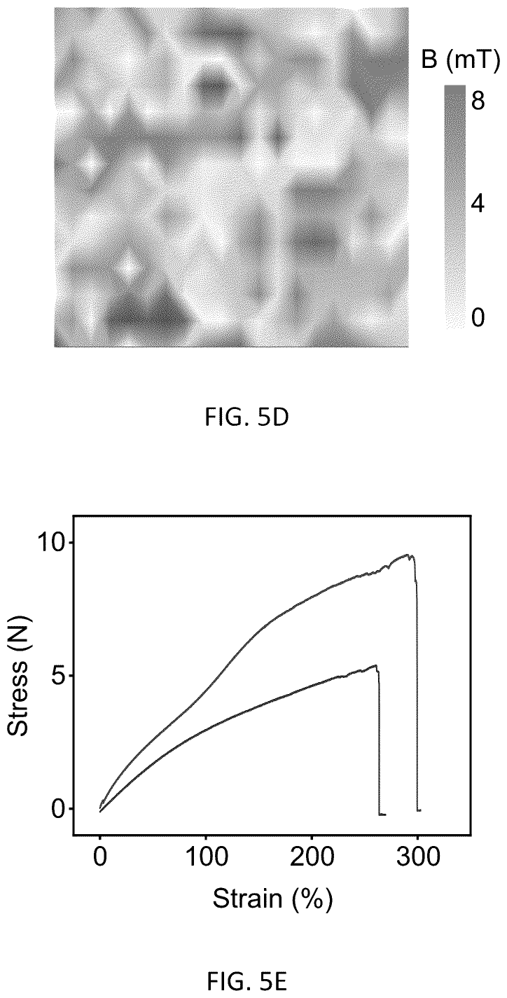

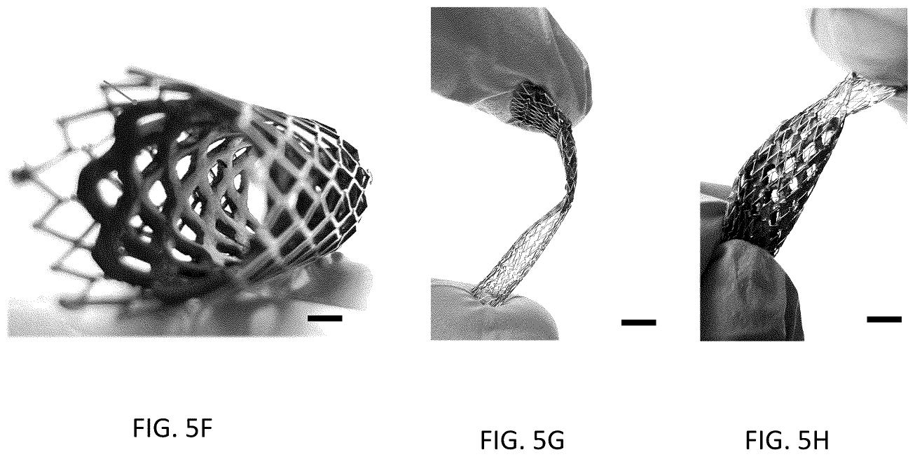

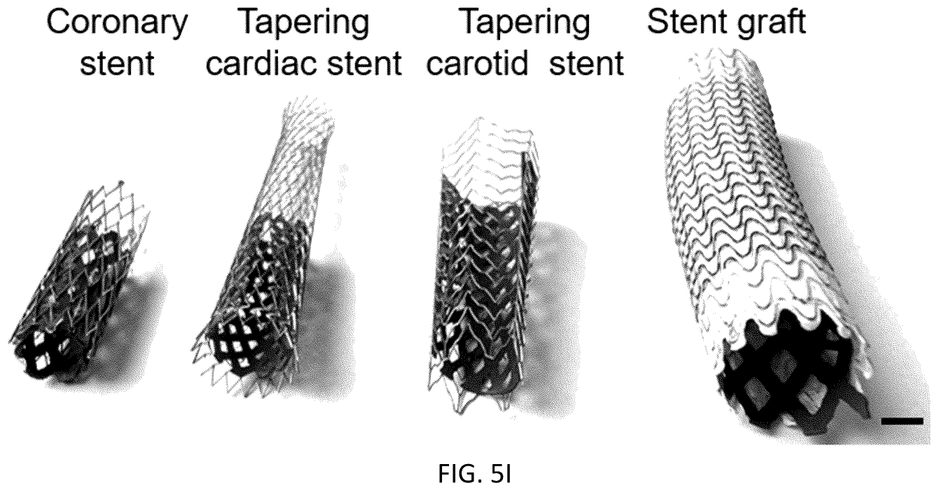

To test the mechanisms utilized in the system 100 experimentally, a soft magnetoelastic generator (MEG) was developed as a pressure sensor to wrap around a carotid artery of a Yorkshire swine in vitro to monitor simulated blood flow wirelessly (FIG. 3A). The soft MEG was comprised of an 800 μm magnetoelastic thin film (Ecoflex rubber/NdFeB nanoparticles), which can be easily wrapped around arteries of various sizes. This soft MEG demonstrated stretchability up to 500%, high sensitivity of 80 nA/kPa in the range of 0-20 kPa (corresponding to blood pressure of 0-150 mmHg), a rapid response time of ˜15 ms, a high signal-to-noise ratio (SNR) of 52 dB, and excellent cycling stability,¹⁵⁻¹⁷ which are better than previously reported cuff-type flow sensors.¹⁹ In vivo pilot studies were performed in rats to demonstrate the biocompatibility of the soft MEG. The biocompatible silicone (FIG. 5B, control) and the device (FIG. 5C) were implanted in rats for 14 days and the results indicate that there was no obvious inflammatory reaction. Then, a computer-regulated syringe pump (Harvard PHD 2000) was used to pump the water flow through the swine carotid artery. Due to the pulsatile nature of blood flow, the artery experiences stereotypical changes in vessel diameter repeatedly over time that deformed the magnetoelastic thin film and shifted its magnetic flux, thus inducing a current in the reader coil (FIG. 5D). Meanwhile, an occlusion was also experimentally induced in the vessel by mechanically clamping the carotid artery, and the real time current signals generated by the soft MEG revealed the variation in the blood flow (FIG. 5E). All considered, the preliminary results confirm the feasibility of the system 100 and demonstrate the system's advantages over traditional flow sensors such as the high sensitivity and accuracy, fast response times, excellent cycling stability, and biocompatibility.

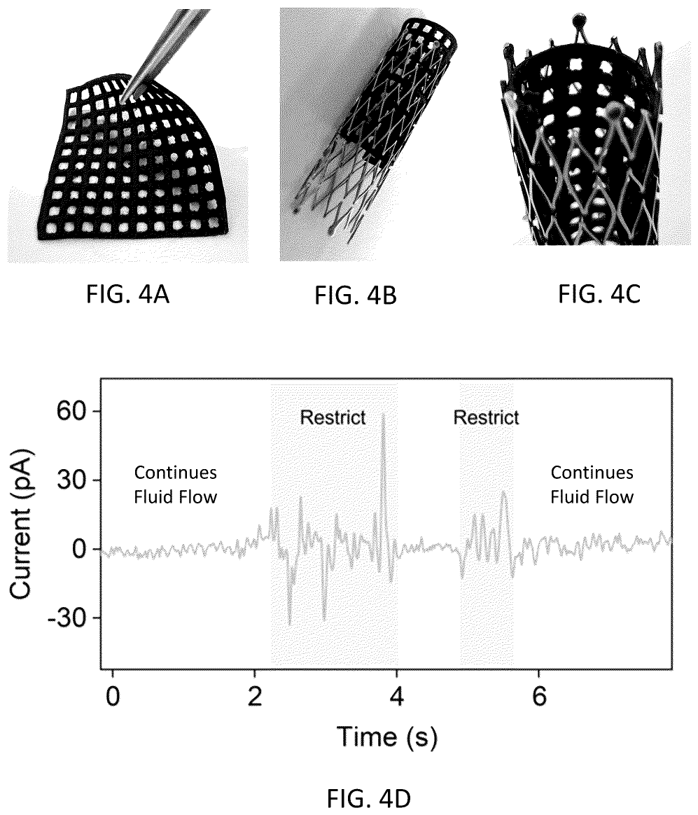

In another experiment, a biocompatible magnetoelastic micromesh (BMM) was developed with a dimension of 15 mm by 15 mm by 0.1 mm and comprised of an Ecoflex rubber/NdFeB nanoparticle composite by using a laser cuter (FIG. 4A). The BMM was then inserted into the inner wall of a metal lattice to construct a bioelectronic stent sensor (25 mm length, 5.6 mm diameter, and 0.3 mm thickness) (FIGS. 4B-4C). The bioelectronic stent sensor was inserted into a carotid artery of a Yorkshire swine in vitro to monitor simulated blood flow. Due to the pulsatile nature of blood flow, the artery experiences stereotypical changes in vessel diameter repeatedly over time that deformed the BMM and shifted its magnetic flux, thus inducing a current in the reader coil. Meanwhile, an occlusion was also experimentally induced in the vessel by mechanically clamping the carotid artery, and the real time current signals generated by the bioelectronic stent sensor revealed the variation in the blood flow (FIG. 4D).

In another experiment, smart stents were fabricated as shown in FIGS. 5A-5C. A 3D printer with a 100 μm nozzle was used to print a tube with a wall thickness of 200 μm by using Ecoflex silicone/NdFeB (5 μm) ink (FIG. 5A). Then a laser cutter (UNIVERSAL PLS6.150D) was used to remove excess material from the parent tube to create the desired pressure sensitive micromesh design and shape (FIGS. 5B-5C). The as-fabricated pressure sensitive micromesh demonstrated a high magnetic flux density of as high as 8 mT (FIG. 5D), a good stretchability of as high as 300% (FG. 5E), and a low Young's Modules of less than 50 kPa. Second, the optimized BMM was inserted into the inner wall of a metal stent to construct a smart stent. Micro-weld technology and medical instant adhesive were used to securely bond the pressure sensitive micromesh and metal stent on the peaks of crowns (FIG. 5F). The resulting smart stent with a stable adhesion could be bent or twisted without causing any separation (FIGS. 5G-5H), which can withstand deformation without dislodging the pressure sensitive micromesh during device delivery as well as the blood interactions at high flow conditions. In addition, with a simple reconfiguration, e.g., changing the diameter of stents and crown structures, the devices can be adapted to function on different blood vessels (FIG. 5I), such as coronary stents, tapering cardiac stents, tapering carotid stents, and stent grafts. These fabrication methods are widely used in the stent manufacturing industry, ensuring their compatibility with future clinical settings.

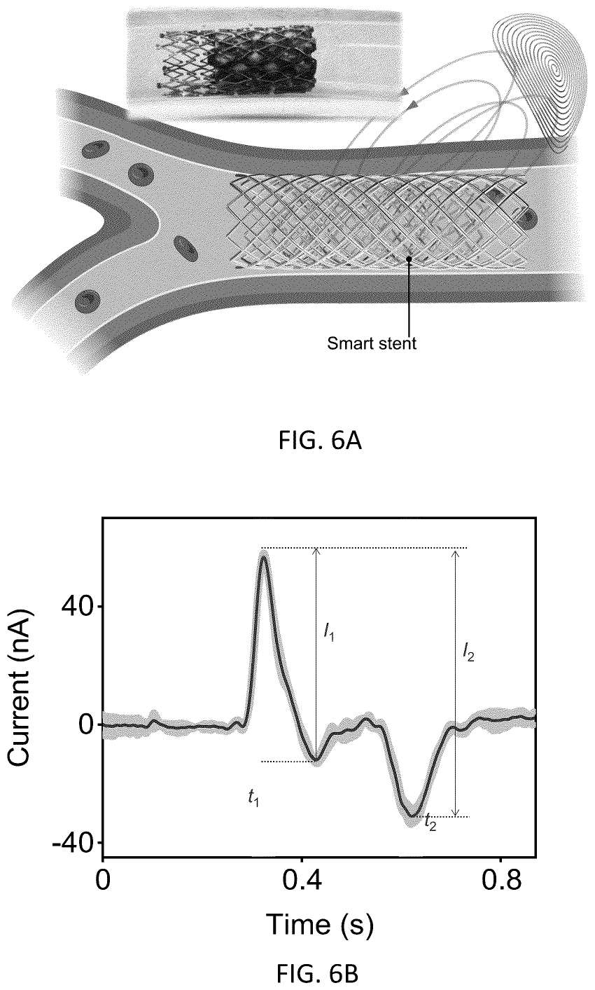

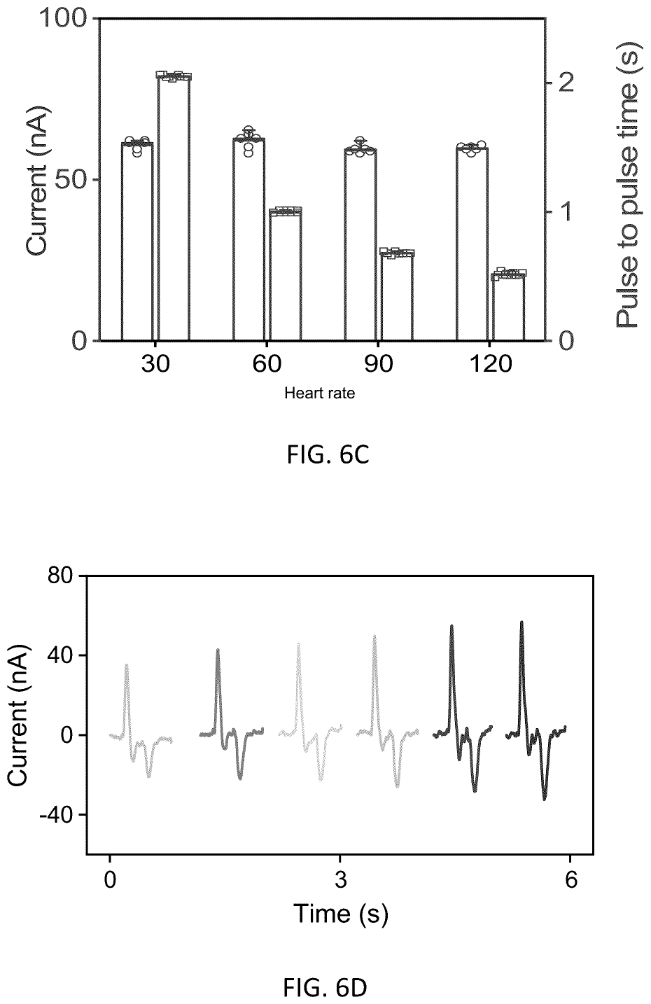

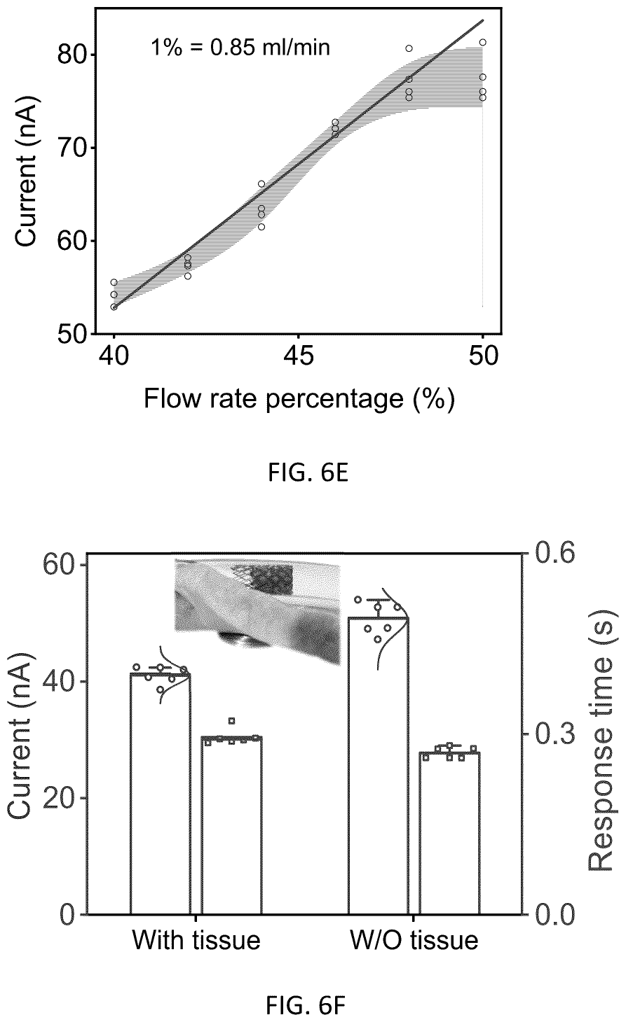

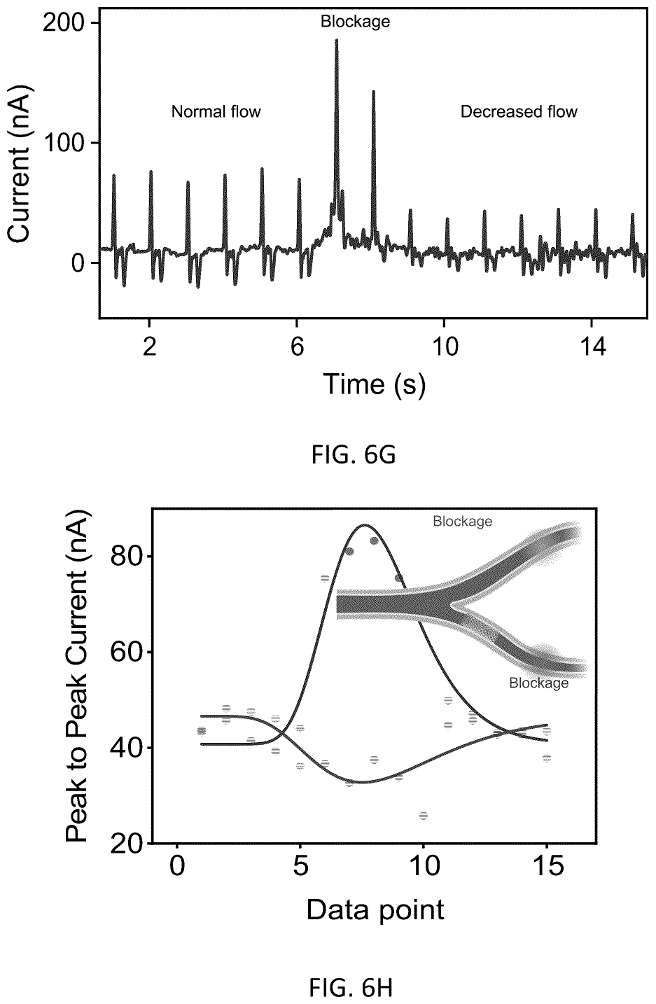

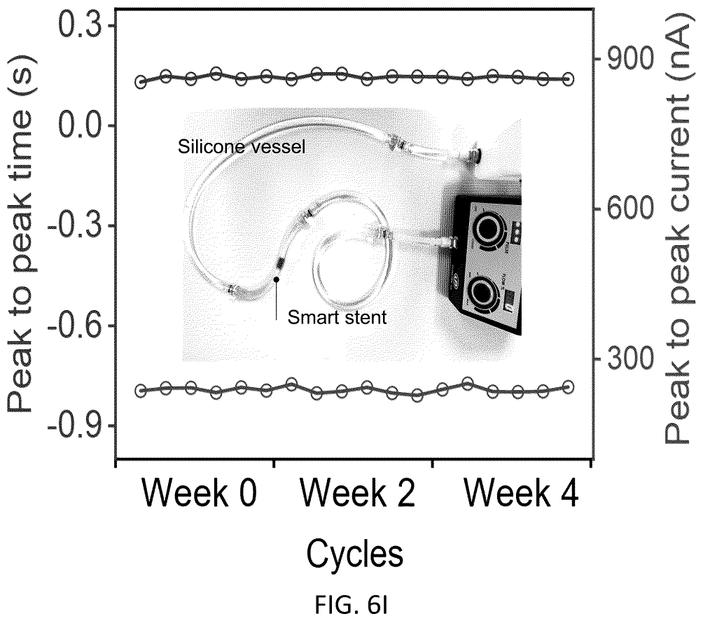

The hemodynamic sensing performance of the as-fabricated smart stent was characterized by using silicone vessels and a pulsatile pump (Flowtek 125) (FIG. 6A). Due to the pulsatile nature of blood flow, the artery experiences stereotypical changes in vessel diameter repeatedly over time, deforming the micromesh and resulting in a shift in its magnetic flux. This flux shift, in turn, induces current signals that were read out with an external coil receiver (20 mm by 10 mm by 1 mm, 200 turns of coils) and a low-noise current preamplifier (Stanford SR570) (FIG. 6B). The changed heartbeat (pulse wave) can be detected by the smart stent (FIG. 6C) by calculating the different pulse to pulse time. Meanwhile, the induced current signals demonstrated a well-linear relationship with flow rates (FIG. 6D), indicating a resolution as low as 0.85 ml/min (FIG. 6E). Further, the transdermal sensing performance was evaluated by using a 2 cm thick porcine tissue to separate the silicone vessels (with deployed stent) and the external coil receiver. The results show that static magnetic fields are transparent to the tissue and can effectively reach the receivers with minor attenuation (FIG. 6F). A blockage was also experimentally induced in the vessel by mechanically clamping the silicon vessel branches (FIG. 6G). When the smart stent located branch was blocked, the decreased flow induced lower current signal (FIG. 6H, red). Meanwhile, when another branch got blocked, the increased flow caused a larger current signal (FIG. 6H, blue). The real-time current signals generated by the smart stent revealed the variation in the fluid flow. Finally, the smart stent was placed into a loop tubing and deionized water was pumped through the loop with a fixed pulse rate of 4.5 Hz for 7 days. The device was cycled for 2.7×10⁶ times in total, equivalent to approximately 4 weeks of heart beating for a healthy adult. There was no obvious attenuation in its sensing performance after long-term pulsatile interactions (FIG. 6I).

In another experiment, the bioelectronic stent sensor was fixed inside a carotid artery model in vitro for flow sensing standard performance testing. Blood simulant solution (Mentice, SIMulant Blood) was pumped through the artery at rates from 10 ml/min-50 ml/min (similar to the real blood flow rate of 33±9.6 ml/min),²³ deforming the BMM and inducing current signals in the stent, which were read out with a low-noise current preamplifier (Stanford SR570). The sensitivity, flow rate detection limits, response time, SNR, and cycling stability were measured to evaluate the sensing performance.

To translate this technology from bench to bedside, the bioelectronic stent sensor device 101 should be compatible with the standard stent placement procedure. To test this, an optimal bioelectronic stent sensor was mounted onto the delivery end of a commercial stent delivery catheter system (without the commercial stent). Scanning electron microscopy (SEM) was used to image the microstructures as well as the nanoparticle distribution inside the BMM thin films. To identify the microstructured magnetoelastic thin film with the best magnetomechanical coupling efficiency, a pressure from 1 mmHg to 200 mmHg was applied to the films to assess the corresponding magnetic field variations. Microstructured magnetoelastic thin films with the optimal magnetomechanical coupling factor, such as larger than 1×10⁻⁸ T/Pa for example, were selected. A laser cutter (such as UNIVERSAL PLS6.150D) was used to cut the optimal thin film into a micromesh structure and get the BMM with enhanced sensitivity and response time.

The catheter system was advanced per routine into the target silicone vessels of the vascular flow system and the bioelectronic stent sensor centered across the target area/lesion. The outer catheter of the delivery system was unsheathed per routine, allowing the stent to open and implant. Clinically relevant post-implantation balloon angioplasty was performed to test the rigor and durability of the bioelectronic stent sensor. To reflect clinical conditions, carotid artery silicone vessels replicated from real atherosclerosis patient anatomy was used to test the device delivery.²⁴ The novel dual-layered construction, small footprint, low profile, and high mechanical robustness of the bioelectronic stent sensors enables smooth insertion with minimized guiding resistance and no apparent damage both mechanically and electrically.

The deployed bioelectronic stent sensor was used to convert the fluid flow into high-fidelity electrical signals for flow rate measurements. A realistically beating 3D heart model of the vascular flow system was used to produce physiologic pulsatile flow waveforms through the silicone vessels.²⁴ The physiological flows were manipulated with a rate range of 1-20 cm/s to deform the bioelectronic stent sensor, which generated the corresponding current signals. Devices deployed in silicone vessels of various diameters and levels of tortuosity were tested. Finally, finite element analysis (FEA) was used to establish an empirical relationship between the readout current signals (nA) and the set value of flow rate (cm/second).

The hemocompatibility of the bioelectronic stent sensor was evaluated in a widely used human blood flow loop model.²⁵ A bioelectronic stent sensor device 101 was placed into the lumen of the loop tubing (4× loops). Four empty loops and four loops with traditional bare-metal stents served as negative controls. Heparinized whole blood collected from healthy adult human volunteers was pumped through these loops with an average flow rate of 100 mL/min inside a 37° C. chamber for 60 min. After the experiment, the bioelectric stents and blood from each loop were collected for analysis. Thrombin generation, platelet activation, and thrombus visualization (through SEM) were the outputs.²⁶

Based on preliminary data from in vitro and in vivo biocompatibility studies, bioelectronic stent sensors preserve the hemocompatibility safety and demonstrate no statistical significance differences from the negative control.

An experiment was performed to demonstrate the feasibility of dynamic blood flow rate monitoring in a swine model in vivo using the proposed bioelectronic stent sensor device 101. The experiment used active protocol for surgical and endovascular access to swine vasculature (ARC Protocol pending) in the TRIC. Yorkshire swine (Sus scrofa) of either gender between 40-50 kg were used in this test. Swine were pre-medicated with aspirin 325 mg daily and clopidogrel 75 mg daily per routine for endovascular stent implantation. During surgery, the swine were anesthetized, and vital signs (heart rate, blood pressure, oxygenation, and temperature) were monitored continuously per routine. A standard catheter was used to deliver and to deploy the sterilized bioelectronic stent sensor device to the common carotid artery under real-time fluoroscopy. It was envisioned that the postoperative blood flow of the swine will deform the bioelectronic stent sensor and generate current signals correspondingly, which will be used to acquire the blood flow rate from the empirical relationship. Meanwhile, a Transonic Flow Probe (Transonic HVT100) was used to measure the real-time blood flow rate as the real values. The measured results were compared from the bioelectronic stent sensor with the “gold standard” values by using Bland-Altman analysis to evaluate bias and agreement: tolerability index (ATI).

To test the ability of the bioelectronic stent sensor device 101 for in-stent restenosis detection and management, the blood flow was blocked, and vessel occlusion was induced on the same swine model. First, an outward tourniquet was applied around the segment of the vessel with the stent and then tourniquet was tightened to cause increasing amounts of vessel stenosis (narrowing). To quantify the degree of the vessel occlusion, the vessel diameter was measured using digital subtraction angiography (DSA) before and after the tourniquet was tightened. The segment diameter was controlled as 25%, 50%, and 75% of the normal caliber distal vessel, respectively. The bioelectronic stent sensor device 101 was used to monitor the blood flow under these conditions. It was envisioned that when the blood flow is blocked, intraluminal pressure experienced by the bioelectronic stent sensor will decrease the generated current signals. The calculated blood flow rate under the induced vessel occlusion was compared with measured blood flow rates of the normal status by using the student's t-test. A p-value of 0.05 or less would indicate a significant difference between healthy and vessel occlusion conditions.

No iatrogenic injury in the swine model was observed, and the bioelectronic stent sensor successfully monitored the blood flow rate in vivo and distinguished the blood flow rate variation under the experimentally induced vessel occlusion. One potential pitfall was the baseline signal drift during the animal test. To address this issue, a signal processing algorithm was developed to examine the mean of the absolute values of the differential signals over each interval via filtering, which normalize the device sensitivity and reduced signal fluctuations according to the different flow rates. Another potential pitfall was that forces applied externally to the vessel segment with the stent to create in-stent stenosis may not have been sufficient to alter blood flow. To address this issue, an endovascular balloon catheter was additionally placed and inflated inside the artery proximal to the stent and/or inside the stent to further reduce anterograde blood flow reaching the stent.

General statistical analyses were used for experimental design and data analysis. Specifically, FEA was used to establish an empirical relationship between the readout current signals and the flow rate. The Bland-Altman analysis was used to evaluate bias and ATI of the bioelectronic stent sensor measured blood flow rates and the set values. The student's t-test was used for comparing the measured blood flow rates of the healthy state versus vessel occlusion condition. All data analyses were performed in a blinded fashion with the collaboration of team members. The observers doing data analysis were blinded to the experimental group information.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g., a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

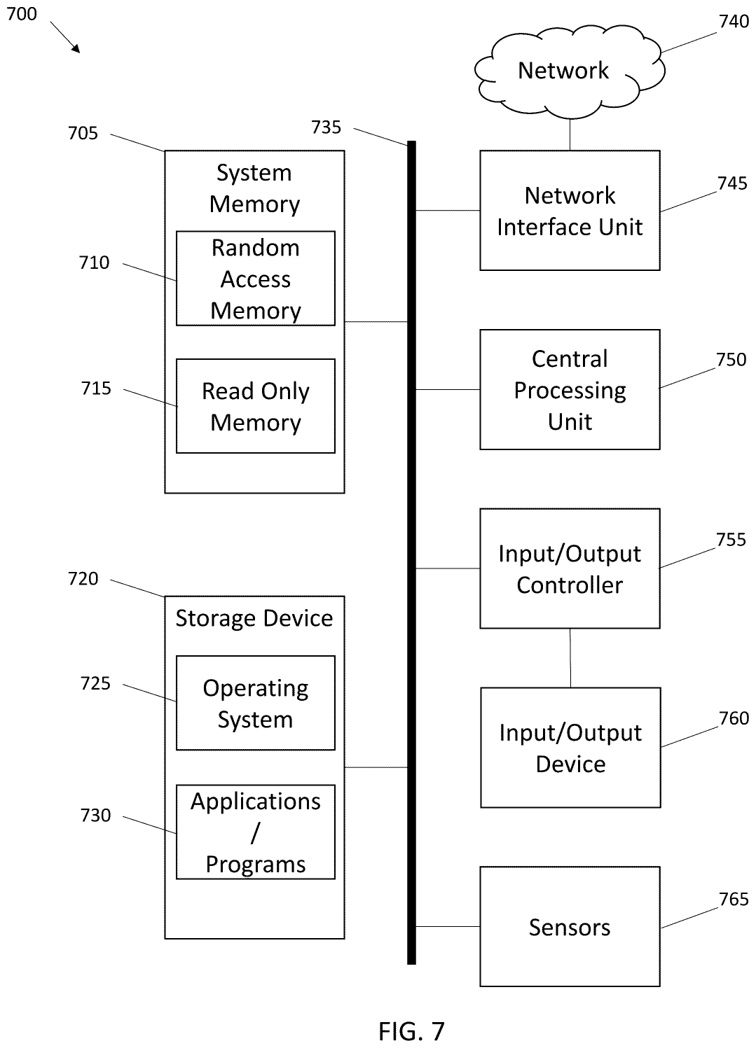

FIG. 7 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 7 depicts an illustrative computer architecture for a computer 700 for practicing the various embodiments of the invention. The computer architecture shown in FIG. 7 illustrates a conventional personal computer, including a central processing unit 750 (“CPU”), a system memory 705, including a random-access memory 710 (“RAM”) and a read-only memory (“ROM”) 715, and a system bus 735 that couples the system memory 705 to the CPU 750. A basic input/output system containing the basic routines that help to transfer information between elements within the computer, such as during startup, is stored in the ROM 715. The computer 700 further includes a storage device 720 for storing an operating system 725, application/program 730, and data.

The storage device 720 is connected to the CPU 750 through a storage controller (not shown) connected to the bus 735. The storage device 720 and its associated computer-readable media, provide non-volatile storage for the computer 700. Although the description of computer-readable media contained herein refers to a storage device, such as a hard disk or CD-ROM drive, it should be appreciated by those skilled in the art that computer-readable media can be any available media that can be accessed by the computer 700.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 700 may operate in a networked environment using logical connections to remote computers through a network 740, such as TCP/IP network such as the Internet or an intranet. The computer 700 may connect to the network 740 through a network interface unit 745 connected to the bus 735. It should be appreciated that the network interface unit 745 may also be utilized to connect to other types of networks and remote computer systems.

The computer 700 may also include an input/output controller 755 for receiving and processing input from a number of input/output devices 760, including a keyboard, a mouse, a touchscreen, a camera, a microphone, a controller, a joystick, or other type of input device. Similarly, the input/output controller 755 may provide output to a display screen, a printer, a speaker, or other type of output device. The computer 700 can connect to the input/output device 760 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 720 and RAM 710 of the computer 700, including an operating system 725 suitable for controlling the operation of a networked computer. The storage device 720 and RAM 710 may also store one or more applications/programs 730. In particular, the storage device 720 and RAM 710 may store an application/program 730 for providing a variety of functionalities to a user. For instance, the application/program 730 may comprise many types of programs such as a word processing application, a spreadsheet application, a desktop publishing application, a database application, a gaming application, internet browsing application, electronic mail application, messaging application, and the like. According to an embodiment of the present invention, the application/program 730 comprises a multiple functionality software application for providing word processing functionality, slide presentation functionality, spreadsheet functionality, database functionality and the like.

The computer 700 in some embodiments can include a variety of sensors 765 for monitoring the environment surrounding and the environment internal to the computer 700. These sensors 765 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The following publications are each hereby incorporated herein by reference in their entirety:
1. Libby, P., The changing landscape of atherosclerosis. Nature 2021, 592 (7855), 524-533.
2. Byrne, R. A.; Stone, G. W.; Ormiston, J.; Kastrati, A., Coronary balloon angioplasty, stents, and scaffolds. The Lancet 2017, 390 (10096), 781-792.
3. Boudoulas, K. D.; Triposkiadis, F.; Geleris, P.; Boudoulas, H., Coronary Atherosclerosis: Pathophysiologic Basis for Diagnosis and Management. Prog. Cardiovasc. Dis. 2016, 58 (6), 676-692.
4. Yahagi, K.; Kolodgie, F. D.; Otsuka, F.; Finn, A. V.; Davis, H. R.; Joner, M.; Virmani, R., Pathophysiology of native coronary, vein graft, and in-stent atherosclerosis. Nat. Rev. Cardiol. 2016, 13 (2), 79-98.
5. Kim, M. S.; Dean, L. S., In-Stent Restenosis. Cardiovasc. Ther. 2011, 29 (3), 190-198.
6. Andreini, D.; Mushtaq, S.; Pontone, G.; Conte, E.; Collet, C.; Sonck, J.; D'Errico, A.; Odoardo, L. D.; Guglielmo, M.; Baggiano, A.; Trabattoni, D.; Ravagnani, P.; Montorsi, P.; Teruzzi, G.; Olivares, P.; Fabbiocchi, F.; Martini, S. D.; Calligaris, G.; Annoni, A.; Mancini, M. E.; Formenti, A.; Magatelli, M.; Consiglio, E.; Muscogiuri, G.; Lombardi, F.; Fiorentini, C.; Bartorelli, A. L.; Pepi, M., CT Perfusion Versus Coronary CT Angiography in Patients With Suspected In-Stent Restenosis or CAD Progression. JACC Cardiovasc. Imaging 2020, 13 (3), 732-742.
7. Green, J.; Ryer, E.; Borden, N.; Ali, B.; Garvin, R.; Yang, A.; Hashmi, A.; Salzler, G.; Elmore, J., Defining Duplex Ultrasound Criteria for In-Stent Restenosis of the Superior Mesenteric Artery. Ann. Vasc. Surg. 2021, 74, 294-300.
8. Siontis, G. C. M.; Stefanini, G. G.; Mavridis, D.; Siontis, K. C.; Alfonso, F.; Pérez-Vizcayno, M. J.; Byrne, R. A.; Kastrati, A.; Meier, B.; Salanti, G.; Jüni, P.; Windecker, S., Percutaneous coronary interventional strategies for treatment of in-stent restenosis: a network meta-analysis. The Lancet 2015, 386 (9994), 655-664.
9. Hoare, D.; Bussooa, A.; Neale, S.; Mirzai, N.; Mercer, J., The Future of Cardiovascular Stents: Bioresorbable and Integrated Biosensor Technology. Adv. Sci. 2019, 6 (20), 1900856.
10. Son, D.; Lee, J.; Lee, D. J.; Ghaffari, R.; Yun, S.; Kim, S. J.; Lee, J. E.; Cho, H. R.; Yoon, S.; Yang, S.; Lee, S.; Qiao, S.; Ling, D.; Shin, S.; Song, J.-K.; Kim, J.; Kim, T.; Lee, H.; Kim, J.; Soh, M.; Lee, N.; Hwang, C. S.; Nam, S.; Lu, N.; Hyeon, T.; Choi, S. H.; Kim, D.-H., Bioresorbable Electronic Stent Integrated with Therapeutic Nanoparticles for Endovascular Diseases. ACS Nano 2015, 9 (6), 5937-5946.
11. Herbert, R.; Lim, H.-R.; Rigo, B.; Yeo, W.-H., Fully implantable wireless batteryless vascular electronics with printed soft sensors for multiplex sensing of hemodynamics. Sci. Adv. 2022, 8 (19), eabm1175.
12. Chen, X.; Assadsangabi, B.; Hsiang, Y.; Takahata, K., Enabling Angioplasty-Ready “Smart” Stents to Detect In-Stent Restenosis and Occlusion. Adv. Sci. 2018, 5 (5), 1700560.
13. Herbert, R.; Mishra, S.; Lim, H.-R.; Yoo, H.; Yeo, W.-H., Fully Printed, Wireless, Stretchable Implantable Biosystem toward Batteryless, Real-Time Monitoring of Cerebral Aneurysm Hemodynamics. Adv. Sci. 2019, 6 (18), 1901034.
14. Howe, C.; Mishra, S.; Kim, Y.-S.; Chen, Y.; Ye, S.-H.; Wagner, W. R.; Jeong, J.-W.; Byun, H.-S.; Kim, J.-H.; Chun, Y.; Yeo, W.-H., Stretchable, Implantable, Nanostructured Flow-Diverter System for Quantification of Intra-aneurysmal Hemodynamics. ACS Nano 2018, 12 (8), 8706-8716.
15. Zhao, X.; Zhou, Y.; Xu, J.; Chen, G.; Fang, Y.; Tat, T.; Chen, J., Soft fibers with magnetoelasticity for wearable electronics. Nat. Commun. 2021, 12, 6839.
16. Chen, G.; Zhao, X.; Andalib, S.; Xu, J.; Zhou, Y.; Tat, T.; Lin, K.; Chen, J., Discovering giant magnetoelasticity in soft matter for electronic textiles. Matter 2021, 4 (10), 3725-3740.
17. Zhao, X.; Chen, G.; Zhou, Y.; Nashalian, A.; Xu, J.; Tat, T.; Song, Y.; Libanori, A.; Xu, S.; Li, S.; Chen, J., Giant Magnetoelastic Effect Enabled Stretchable Sensor for Self-Powered Biomonitoring. ACS Nano 2022, 16 (4), 6013-6022.
18. Chiang, N.; Scarabelli, L.; Vinnacombe-Willson, G. A.; Perez, L. A.; Dore, C.; Mihi, A.; Jonas, S. J.; Weiss, P. S., Large-Scale Soft-Lithographic Patterning of Plasmonic Nanoparticles. ACS Mater. Lett. 2021, 3 (3), 282-289.
19. Liu, W.; Wang, J.; Xu, X.; Zhao, C.; Xu, X.; Weiss, P. S., Single-Step Dual-Layer Photolithography for Tunable and Scalable Nanopatterning. ACS Nano 2021, 15 (7), 12180-12188.
20. Xu, X.; Liu, W.; Ji, Z.; Hao, D.; Yan, W.; Ye, Z.; Hu, Y.; Fang, M.; Wang, C.; Ma, L.; Huang, J.; Xu, X.; Weiss, P. S., Large-Area Periodic Organic-Inorganic Hybrid Perovskite Nanopyramid Arrays for High-Performance Photodetector and Image Sensor Applications. ACS Mater. Lett. 2021, 3 (8), 1189-1196.
21. Musiałek, P.; Roubin, G. S., Commentary: Double-Layer Carotid Stents: From the Clinical Need, through a Stent-in-Stent Strategy, to Effective Plaque Isolation . . . the Journey Toward Safe Carotid Revascularization Using the Endovascular Route. J. Endovasc. Ther. 2019, 26 (4), 572-577.
22. Boutry, C. M.; Beker, L.; Kaizawa, Y.; Vassos, C.; Tran, H.; Hinckley, A. C.; Pfattner, R.; Niu, S.; Li, J.; Claverie, J.; Wang, Z.; Chang, J.; Fox, P. M.; Bao, Z., Biodegradable and flexible arterial-pulse sensor for the wireless monitoring of blood flow. Nat. Biomed. Eng. 2019, 3 (1), 47-57.
23. Riva, C. E.; Grunwald, J. E.; Sinclair, S. H.; Petrig, B. L., Blood velocity and volumetric flow rate in human retinal vessels. Invest. Ophthalmol. Vis. Sci. 1985, 26 (8), 1124-1132.
24. Kaneko, N.; Mashiko, T.; Ohnishi, T.; Ohta, M.; Namba, K.; Watanabe, E.; Kawai, K., Manufacture of patient-specific vascular replicas for endovascular simulation using fast, low-cost method. Sci. Rep. 2016, 6 (1), 39168.
25. Girdhar, G.; Andersen, A.; Pangerl, E.; Jahanbekam, R.; Ubl, S.; Nguyen, K.; Wainwright, J.; Wolf, M. F., Thrombogenicity assessment of Pipeline Flex, Pipeline Shield, and FRED flow diverters in an in vitro human blood physiological flow loop model. J. Biomed. Mater. Res. 2018, 106 (12), 3195-3202.
26 Starke, R. M.; Thompson, J.; Pagani, A.; Choubey, A.; Wainwright, J. M.; Wolf, M. F.; Jahanbekam, R.; Girdhar, G., Preclinical safety and efficacy evaluation of the Pipeline Vantage Embolization Device with Shield Technology. J. Neurointerv. Surg. 2020, 12 (10), 981-986.
27. Y. Zhou; X. Zhao; J. Xu; Y. Fang; G. Chen; Y. Song; S. Li; J. Chen. Giant Magnetoelastic Effect in Soft Systems for Bioelectronics. Nat. Mater. 2021, 20 (12), 1670-1676.
28. A. Libanori; G. Chen; X. Zhao; Y. Zhou; J. Chen. Smart Textiles for Personalized Healthcare. Nat. Electron. 2022, 5 (3), 142-156.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A bioelectronic stent sensor device, comprising:
a first hollow cylindrical lattice; and
a second hollow cylindrical lattice attached to a first surface of the first lattice, comprising a biocompatible magnetoelastic micromesh (BMM).

2. The device of claim 1, wherein the first surface of the first lattice comprises an inner surface.

3. The device of claim 1, wherein the first lattice comprises a metal lattice.

4. The device of claim 4, wherein the metal lattice includes a layer of nanoparticles, metal nanoparticles, noble metal nanoparticles, or gold nanoparticles.

5. The device of claim 1, wherein the BMM comprises a plurality of nanomagnets embedded in a polymer matrix.

6. The device of claim 1, wherein the second lattice includes a plurality of microstructures in an array.

7. The device of claim 6, wherein the microstructures comprise pyramids, cylinders, or hemispheres.

8. The device of claim 6, wherein the microstructures have a lateral length of 100 nm to 5000 nm, and a pitch of 1 μm to 1000 μm.

9. The device of claim 6, wherein the array is lithographically patterned.

10. The device of claim 1, further comprising an antenna attached to the first lattice.

11. The device of claim 10, wherein the antenna is attached via laser microwelding.

12. The device of claim 1, wherein the second lattice is attached to the first lattice via a cyanoacrylate instant adhesive or a catechol-based adhesive.

13. The device of claim 1, wherein the first lattice comprises a double layer lattice, and wherein the second lattice is anchored between the layers of the first lattice.

14. The device of claim 1, wherein the BMM is configured to deform and shift its magnetic flux to induce a current in the first lattice.

15. The device of claim 1, wherein the BMM comprises a polymer and magnetic nanoparticle composite.

16. The device of claim 15, wherein the polymer comprises an Ecoflex rubber and the magnetic nanoparticle composite comprises NdFeB nanoparticles.

17. The device of claim 15, wherein a nanoparticle layer surrounds the magnetic nanoparticle composite.

18. The device of claim 17, wherein the surrounding nanoparticle layer comprises SiO₂ nanoparticles.

19. The device of claim 1, wherein the device has at least one of a sensitivity detection limit of less than 0.5 cm/second, a low flow rate detection limit of less than 0.5 cm/second, a short response time of less than 15 ms, a high signal-to-noise ratio (SNR) of greater than 50 dB, and long-term stability.

20. A bioelectronic stent sensor system, comprising:
a bioelectronic stent sensor device comprising a first hollow cylindrical lattice, and a second hollow cylindrical lattice attached to a first surface of the first lattice, comprising a biocompatible magnetoelastic micromesh (BMM); and
a computing system communicatively connected to the bioelectronic stent sensor device, comprising a processor and a non-transitory computer-readable medium with instructions stored thereon, which when executed by a processor, perform steps comprising:
receiving readout current signals from the bioelectronic stent sensor device; and
calculating a blood flow rate, a pressure, a pulse rate, an embolic event, a vessel stiffness or change thereof, an oxygenation, or a particle in the flow based on the readout current signals via an established empirical relationship between the readout current signals and a flow rate value.

21. The system of claim 20, wherein the bioelectronic stent sensor device is wirelessly communicatively connected to the computing system via a wireless communication protocol comprising 3G, 4G/LTE, 5G, 6G, Bluetooth, Bluetooth Low Energy (BLE), Zigbee, or near-field communication (NFC).

22. A blood flow rate monitoring method, comprising:
providing the bioelectronic stent sensor system of claim 20;
implanting the bioelectronic stent sensor device via a standard stent placement procedure;
receiving readout current signals from the bioelectronic stent sensor device on the computing system; and
calculating a blood flow rate a pressure, a pulse rate, an embolic event, a vessel stiffness or change thereof, an oxygenation, or a particle in the flow based on the readout current signals via an established empirical relationship between the readout current signals and a flow rate value.

23. The method of claim 22, wherein finite element analysis (FEA) is used to establish the empirical relationship between the readout current signals and the flow rate value.
